(19) United States
(12) Patent Application Publication
(10) Pub. No.: US 2024/0122458 A1
(43) Pub. Date: Apr. 18, 2024

(54) SHAFT FOR A FLEXIBLE ENDOSCOPE OR FOR A FLEIXBLE ENDOSCOPIC INSTRUMENT

(71) Applicant: Karl Storz SE & Co. KG, Tuttlingen (DE)

(72) Inventors: Alexander FUCHS, Tuttlingen (DE); Tobias LOTTES, Helmbrechts (DE)

(73) Assignee: Karl Storz SE & Co. KG, Tuttlingen (DE)

(21) Appl. No.: 18/554,765

(22) PCT Filed: Apr. 11, 2022
(86) PCT No.: PCT/EP2022/059651

§ 371 Date: Oct. 10, 2023

(30) Foreign Application Priority Data
Apr. 12, 2021 (DE) 10 2021 109 022.9

(51) Int. Cl. A61B 1/005; A61B 1/00

(52) U.S. Cl. CPC A61B 1/0055; A61B 1/0011; A61B 1/0057

(57) ABSTRACT

A shaft according to the invention for a flexible endoscope or for a flexible endoscopic instrument includes a first shaft section and a second shaft section arranged on the distal side of the first shaft section, wherein the second shaft section is able to be angled by means of at least one pull element extending inside the second shaft section, wherein the second shaft section has a support structure, and wherein an adapter is arranged between a distal end region of the first shaft section and a proximal end region of the support structure. The invention also relates to a method for assembling a shaft for a flexible endoscope or for a flexible endoscopic instrument, and also to a flexible endoscope and a flexible endoscopic instrument.

### CROSS-REFERENCE TO RELATED APPLICATIONS

This application is the U.S. national stage of PCT/EP2022/059651 filed on Apr. 11, 2022, which claims priority of German Patent Application No. DE 10 2021 109 022.9 filed on Apr. 12, 2021, the contents of which are incorporated herein.

### TECHNICAL FIELD

The present disclosure relates to a shaft for a flexible endoscope or for a flexible endoscopic instrument according to the preamble of claim 1, to a method for assembling such a shaft, and to a flexible endoscope and a flexible endoscopic instrument having such a shaft.

### BACKGROUND

Flexible endoscopes have a flexible, elongate shaft that is suitable for insertion into a cavity, such as a cavity inside the body or a cavity of a technical object. In the region of the distal end of the endoscope shaft (i.e. the end remote from the user), imaging optics are arranged in order to generate an image of a scene within the cavity, in which case, according to a common design, an electronic image recorder is arranged in the distal end region for the purpose of recording the image, the signals of which image recorder are transmitted, via electrical lines running within the shaft, to the proximal end region of the shaft (i.e. the end region near the user). As a rule, the flexible endoscope also comprises an illumination system for illuminating the cavity that is to be observed. Furthermore, the endoscope shaft can contain one or more working channels for the passage of endoscopic working instruments from the proximal to the distal end region of the shaft, and also suction and irrigation channels.

Endoscopic instruments are also known which comprise a flexible, elongate shaft that is also suitable for insertion into such a cavity. Such a flexible endoscopic instrument can be used to carry out manipulations in the cavity and, for this purpose, can be designed for example as a grasping instrument for gripping and manipulating tissue or objects in the cavity inside the body or within the cavity of a technical object. For this purpose, a tool is arranged at the distal end of the flexible shaft and can be operated from the proximal end of the shaft via a transmission means running within the shaft. Such a flexible endoscopic instrument does not generally have its own optics for recording an endoscopic image, but can in particular be used together with a flexible endoscope.

Flexible endoscopes and flexible endoscopic instruments are typically used for observing or performing manipulations in a cavity that can be reached via a curved access path or, for example, to observe the interior of an organ that itself has a curved shape. By virtue of its flexibility, the shaft can adapt to the curvature of the access route or of the organ. However, it is often desirable to be able to actively bend the distal end of the shaft, i.e. the tip of the endoscope or of the endoscopic instrument, in order to facilitate the introduction of the endoscope or of the endoscopic instrument through a channel that does not run in a straight line, in order to be able to move the tip within a cavity in a lateral direction, and to be able to change the viewing direction of the imaging optics or the orientation of a tool. For this purpose, the shaft has a controllable section, in particular a controllable end section, which can be actively angled by a desired amount in a desired direction and can be controlled for this purpose from the proximal end of the endoscope or of the endoscopic instrument.

To permit controllable bending of a shaft of a flexible endoscope or of a flexible endoscopic instrument, it is known to design it with a base structure or support structure made up of links or segments which are arranged one behind the other and pivotably connected to one another by hinges and which can be actuated via pull cables or Bowden cables guided in the endoscope shaft. The bending of the controllable section from a straight shape to a curved or angled shape, and vice versa, can be controlled by axial movement of control wires or pull wires acting in pairs against one another. To actuate the pull wires, handwheels or rotary knobs can be provided which are arranged, for example, on a handle of the endoscope or of the endoscopic instrument. The support structure of the bendable section is typically enclosed by a flexible sleeve, which preferably forms a smooth, continuous surface with a sleeve of the remaining shaft sections.

The support structure can be designed in one piece, with the individual segments being able to be connected to one another by film hinges. Such a one-piece support structure can be produced cost-effectively and is therefore particularly suitable for flexible endoscopes or flexible endoscopic instruments that are intended for single use.

An articulated bending arrangement for a single-use endoscope, which comprises a plurality of interconnected segments, is known for example from U.S. Pat. No. 10,052,013 B2.

DE 41 15 419 A1 discloses a long and thin flexible tube for an endoscope, which forms an outer wall for an insertion part and which receives an installation element, and a method for producing the tube, wherein the installation element is inserted into a plurality of short flexible tube sections, as long as these are still separated from one another, and two adjacent short tube sections into which the installation element has been inserted are connected to each other by means of associated coupling means.

According to DE 103 08 902 A1, a flexible ureteropyeloscope comprises a shaft which has a front end with a first and a second active deflection region which are connected in series and which have a first and a second frame element with shape memory, wherein the first and the second frame element are connected to each other via a connecting piece, and one end of an operating wire is firmly connected to the connection piece starting from an operating region.

DE 43 20 018 A1 discloses an endoscope with a controllable tip, which endoscope consists of a rigid proximal shaft part and a flexible distal shaft part, with a semi-flexible buffer being arranged in the region of the transition from the rigid to the flexible shaft part.

DE 10 2013 226 591 A1 describes a device and a method for producing an elongate hollow profile element, which forms a jacket element of a bending unit for an endoscope, and inside which a plurality of pull cables run. On the proximal side of the elongate hollow profile element, an intermediate piece is arranged which serves for anchoring the cable pull spiral sleeves of the pull cables.

A flexible endoscope with an angled end section that has a one-piece support structure is known from WO 2014/106511 A1. A distal end segment, a proximal end segment and the intermediate middle segments are designed as a one-piece component. The individual segments are connected to one another by flexible hinge means. The distal end segment comprises a camera and light-emitting diodes. The proximal end segment has, in its surface, a plurality of incisions which support the connection of the bendable section to an insertion tube of the endoscope. The middle segments have a plurality of passages. Two pull wires extend through two passages which are arranged symmetrically on both sides of a plane of the flexible hinge means. The ends of the pull wires are attached in the distal end region and are connected to a control lever in an actuating handle.

A shaft for a flexible endoscope or for an endoscopic instrument, which comprises at least one bendable shaft section with a support structure that comprises a distal end member, a proximal end member and at least a first and a second intermediate member between these, is disclosed in the German patent application DE 10 2020 134 603.4 (not a prior publication) (internal file number P20043DE). The first and second intermediate members are designed differently from each other and are connected to each other in one piece by at least two opposite webs, which define an axis of rotation about which the first and second intermediate members are bendable. A camera head can be arranged on the distal end member, and the proximal end member can be connected to a further shaft section in a force-fitting and/or form-fitting manner.

It has been shown that, in the case of the known shafts for flexible endoscopes or flexible endoscopic instruments, assembly is not optimal. In particular, the connection of the Bowden cables for bending the controllable section and/or adhesive processes during the production of the controllable section or when connecting the controllable section to a proximal section of the shaft and to a camera head or an end effector are complex and, in principle, prone to errors.

### SUMMARY

It is an object of the present disclosure to provide a shaft for a flexible endoscope or for a flexible endoscopic instrument, whereby the disadvantages mentioned above are avoided as far as possible. In particular, it is an object of the disclosure to provide such a shaft which is improved in terms of assembly, in such a way that the connection of the controllable section to a proximal section of the shaft and to a camera head or an end effector is simplified and made possible with increased process reliability. Furthermore, it is an object of the present disclosure to specify a corresponding method for assembling such a shaft and also a flexible endoscope and a flexible endoscopic instrument with such a shaft.

This object is achieved by a shaft according to claim 1 or 12, by a method according to claim 14, and by a flexible endoscope or a flexible endoscopic instrument according to claim 15.

Advantageous developments of the disclosure are set out in the dependent claims.

A shaft according to the disclosure for a flexible endoscope or for a flexible endoscopic instrument is elongate and designed for insertion into a cavity, for example an internal cavity of a human or animal body or a cavity of a technical or other object. The shaft can be designed as a part or assembly of a flexible endoscope or of a flexible endoscopic instrument or can be assembled together with other components to form such an endoscope or instrument. The flexible endoscope can comprise a handle which is arranged at a proximal end of the shaft and which can have operating elements and/or connections for further devices, such as a suction and irrigation device or a camera unit. The flexible endoscopic instrument can have a grip arranged at a proximal end of the shaft and having an actuating element for a tool arranged at the distal end of the shaft. The flexible endoscope or the flexible endoscopic instrument is designed in particular for use in medical, in particular surgical, procedures or for technical applications. Preferably, the shaft is designed for single use, for example for a disposable endoscope or a disposable instrument.

The shaft comprises a first shaft section and a second shaft section which is arranged on the distal side of the first shaft section and which can be angled by means of at least one pull element running within the second shaft section. The second shaft section can therefore be actively angled or controlled, in particular actively curved, while the first shaft section is generally designed to be at least partially flexible, but is not actively curveable. In particular, the second shaft section can be actively angled by a desired amount in a desired direction relative to a distal end region of the first shaft section. The bending can be controlled from the proximal end of the endoscope or of the endoscopic instrument by means of the at least one pull element; in particular, the bending can be controlled via a corresponding actuating element, connected to the at least one pull element, on a handle or a grip arranged at the proximal end of the shaft. Preferably, the second shaft section can be angled by means of two or four pairs of pull elements acting against one another, for example pull wires, which are slidably guided within the shaft and are fixed in the distal end region of the shaft. The second shaft section can be designed as a bendable endoscope tip.

According to the disclosure, the second shaft section has a support structure which extends within the second shaft section in a longitudinal direction of the shaft. The support structure, which can also be referred to as an articulation, can be angled or curved and is formed for this purpose in particular by a plurality of segments or links which follow one another in a longitudinal direction of the shaft and are connected to one another via film hinges. The support structure is preferably formed integrally, in particular in one piece; such an integral support structure can, for example, be made as one piece, or it can also be made by connecting the segments to one another by cohesive bonding. Here and below, the terms “longitudinal axis”, “axial” and “longitudinal direction” refer to a respective section of the shaft, for example to a respective segment of the support structure.

The shaft can comprise further shaft sections. In particular, a rigid end section or a head can be arranged on the distal side of the second shaft section and can be designed, for example, as a camera head in the case of a shaft of an endoscope or as an end effector with a tool in the case of a shaft of an endoscopic instrument. The camera head or the end effector can in particular be connected to the distal end region of the support structure.

Lines and/or channels can run through the first and second shaft sections, for example electrical lines for supplying electronic components of the camera head and for signal transmission, working channels for the passage of working instruments, and also irrigation and/or suction channels or control elements for distally arranged tools. The at least one pull element is preferably routed through the first shaft section as far as the proximal end thereof, where a handle or a grip with an actuating element for controlling the bendable section can be connected to the shaft.

According to the disclosure, an adapter is arranged between a distal end region of the first shaft section and a proximal end region of the support structure. The adapter is thus connected both to the distal end region of the first shaft section and to the proximal end region of the support structure, in particular to a proximal end segment of the support structure. The adapter is preferably designed to be shorter than the first shaft section and shorter than the second shaft section. In particular, the adapter can have a longitudinal extent that corresponds approximately to one to two times the shaft diameter. The first shaft section can have a shaft tube, to the distal end of which the adapter is connected, and the second shaft section can have a cover tube which encloses the support structure, including the at least one pull element, and is connected to the adapter. The shaft tube of the first shaft section and the cover tube of the second shaft section form an outer surface of the shaft, which is preferably designed to transition smoothly from the second shaft section into the first shaft section. The adapter is in particular designed as a separate component, which can be manufactured separately from the preferably integral, in particular one-piece, support structure and separately from components of the first shaft section; alternatively, the adapter can be made in one piece with the support structure and can be produced together with it in a single manufacturing process.

By virtue of the fact that an adapter is interposed between the support structure and the first shaft section, the assembly of the shaft of the endoscope or of the endoscopic instrument can be simplified. In particular, this can make it easier to connect the shaft tube of the first shaft section to the proximal end of the support structure and also to attach the at least one pull element for controlling the bending of the second shaft section. As a result, the process reliability in the manufacture of the shaft can be increased and the corresponding effort reduced, which is particularly advantageous when manufacturing a shaft intended for one-time use.

According to one embodiment of the disclosure, the first shaft section is passively deflectable, for which purpose the first shaft section can be designed to be flexible at least in sections, but not actively curveable or bendable, and instead only bendable by external force, for example by contact with tissue during insertion through a curved access path. Furthermore, according to this embodiment, the support structure of the second shaft section is formed integrally, in particular in one piece, and comprises or is formed by a plurality of segments which follow one another in the longitudinal direction of the shaft and are connected to one another in an articulated manner, for example via film hinges. Such a design can be particularly advantageous in terms of application and operation.

Preferably, the at least one pull element is designed as a pull element of a Bowden cable, in particular as a pull wire or pull cable, which is guided in a Bowden cable sheath in the manner of a Bowden cable, with a distal end of the Bowden cable sheath being fixed in the adapter. The Bowden cable sheath is routed through the first shaft section to the proximal end thereof and can be fixed there or in a handle or a grip that can be connected to the proximal end region of the shaft. The pull wire or the pull cable of the Bowden cable is routed within the Bowden cable sheath through the first shaft section and in or on the support structure through the second shaft section to the distal end thereof. In particular, two or four pull elements guided in pairs opposite each other with respect to a longitudinal axis of the support structure can be provided for controlling the bending of the second shaft section. The bending of the second shaft section can be controlled by axial displacement of the at least one pull element, for example by two pull elements that act against each other being displaced in opposite directions. The distal end of the respective Bowden cable sheaths can be hooked into the adapter, for example. By virtue of the fact that the pull element is routed in the manner of a Bowden cable in a Bowden cable sheath, which is fixed in the adapter, a secure and firm connection of the Bowden cable sheath to the distal end of the first shaft section and to the proximal end of the support structure of the second shaft section can be achieved, and the control of the second shaft section can thereby be improved.

According to a preferred embodiment of the disclosure, the adapter is at least partially annular with an approximately cylindrical outer surface, in particular annularly closed, for example hollow cylindrical, and the cover tube of the second shaft section, in the region of a proximal edge of the approximately cylindrical outer surface, is connected by gluing to the adapter and/or to the first shaft section. A longitudinal axis of the adapter is approximately aligned with a longitudinal axis of the distal end region of the first shaft section and with a longitudinal axis of the proximal end region of the support structure, in particular with a longitudinal axis of the proximal end segment. The cover tube of the second shaft section, which encloses the support structure with the at least one pull element, can also enclose the adapter and, for example, can bear on the outer surface. The cover tube is connected to the adapter and/or to the first shaft section by gluing, with an adhesive point being arranged in the region of the proximal edge of the outer surface of the adapter. The approximately cylindrical outer surface has in particular such an axial extent that a sufficient distance remains between the adhesive point, where an adhesive is applied or introduced, and a distal edge of the outer surface of the adapter, such that no adhesive protrudes over the distal edge of the cylindrical outer surface. The adhesive point can, for example, be a proximal end face of the cover tube that can end flush with the proximal edge of the outer surface of the adapter and that is glued in abutment to a distal end face of a shaft tube of the first shaft section. Alternatively or additionally, the adhesive point can, for example, comprise an annular adhesive surface which forms a proximal edge region of the outer surface of the adapter, and/or the cover tube can be pulled over a distal end region of the shaft tube of the first shaft section and glued there to the latter. In this way, penetration of adhesive into an interior of the adapter or also of a proximal end segment of the support structure can be avoided, where this could lead, for example, to the pull wire or pull cable sticking to the Bowden cable sheath. For this purpose, an axial extent of the preferably closed cylindrical outer surface of the adapter of approximately 2 mm or more is usually sufficient. In this way, secure attachment of the cover tube can be achieved while at the same time increasing process reliability.

According to an advantageous embodiment, the adapter has a proximal section, which is also referred to here as a holding section, which is inserted into a distal end region of the shaft tube of the first shaft section and which is designed for holding the shaft tube. In particular, one or more holding ribs for holding the shaft tube can be arranged on the surface of the holding section. The holding ribs are in particular directed approximately transversely to the longitudinal direction, but can also be arranged obliquely, for example in a helical manner. The holding ribs have a cutting edge directed to the outside, preferably obliquely in the distal direction, which cuts into an inner face of the shaft tube and therefore holds the latter firmly on the adapter. Alternatively, the surface of the holding section can, for example, be designed to be smooth, and the shaft tube is held thereon by friction or by gluing. The holding section is preferably tubular and can, for example, taper slightly conically in the proximal direction from the distal end. The holding section can have one or more longitudinal slots and can therefore be divided into two halves, for example, in order to enable resilient yielding when the shaft tube is pushed on. In this way, a secure connection of the adapter to the shaft tube can be obtained, possibly also without gluing, and thus a secure attachment of the second shaft section to the first shaft section can be achieved. By a suitable choice of the respective external diameters, a smooth and approximately seamless transition between the cover tube of the second shaft section and the shaft tube of the first shaft section can be achieved.

Advantageously, it can be provided that the support structure has a first material and the adapter has a second material, the second material having a greater modulus of elasticity (elastic modulus) than the first material. In particular, it can be provided that the preferably integral, in particular one-piece support structure is produced from and consists of the first material, and that the adapter is produced from and consists of the second material. The adapter is preferably designed in one piece. Since the support structure has to be flexible overall and is designed in particular with film hinges, a material that is flexible and correspondingly has a relatively low modulus of elasticity is advantageous for the support structure. On the other hand, for the use of the endoscope or of the endoscopic instrument, in particular for the insertion into a cavity through a curved access path and for control of the bending, it is advantageous for the shaft tube and thus the first shaft section to be relatively rigid. For the connection of the adapter to the shaft tube of the first shaft section, in particular for ensuring that the adapter exerts a sufficient counterforce upon connection and the shaft tube is held firmly, it is therefore advantageous for the adapter to have a material with a relatively high modulus of elasticity. Preferably, the first and second materials are each a plastic material; this enables cost-effective production. For example, the first material can be a polypropylene with a modulus of elasticity of approximately 1400 MPa, and the second material can be a polycarbonate with a modulus of elasticity of approximately 2400 MPa. By virtue of the fact that the adapter is made of a material with a greater modulus of elasticity than the support structure, it is possible to achieve both an advantageous flexibility of the second shaft section and a secure connection to the stiffer first shaft section.

According to a preferred embodiment of the disclosure, the adapter is connected to a proximal end region of the support structure, for example to the proximal end segment, by means of a latching mechanism, which can comprise at least one latching lug and at least one latching hook interacting with the latter. In particular, the latching lug and the latching hook can be designed to lock through an axial movement of the adapter relative to the proximal end region of the support structure. Preferably, the at least one latching lug and/or the at least one latching hook has a run-on bevel, which enables the latching connection to be closed by an axially directed movement. This allows the assembly of the shaft to be further simplified.

Preferably, the latching hook is approximately L-shaped and has a longitudinal leg directed in particular axially and a transverses leg directed in the circumferential direction of the adapter, wherein the latching lug engages behind the transverse leg in the axial direction and, for connecting the proximal end region of the support structure to the adapter, bears on the transverse leg. Seen in the axial direction, the transverse leg thus forms an undercut, which interacts with an oppositely directed axial end face of the latching lug, which thus likewise forms an undercut. By virtue of the fact that the latching lug bears on the undercut of the transverse leg, it is possible for a relatively large surface area to be made available for fastening the support structure to the adapter, thereby ensuring a particularly strong and secure connection. Furthermore, the latching lug and/or the latching hook preferably have a run-on bevel acting in a radial direction. This makes it possible for a deflection movement of the latching hook and/or of the latching lug to take place in a radial direction upon closure of the latching connection, for example in a radially outward direction; a slight deflection movement may be sufficient and, by means of the large interacting surfaces of the undercut of the transverse leg and the end face of the latching lug, a firm connection of the proximal end region of the support structure to the adapter can still be effected.

Alternatively or in addition to the at least one L-shaped latching hook, at least one latching hook can be provided that is approximately U-shaped, with two approximately axially directed longitudinal legs and one circumferentially directed transverse leg. In particular, such a U-shaped latching hook can be formed when, in the case of two latching hooks which are approximately L-shaped as described above, the transverse legs directed in the circumferential direction of the adapter are connected to each other, at their ends facing away from the respective longitudinal leg, either directly or via a bridge running in the circumferential direction. Even with the approximately U-shaped latching hook, at least one latching lug engages, as described above, in the axial direction behind the transverse leg and bears on the transverse leg in order to connect the proximal end region of the support structure to the adapter, the transverse leg and the axial end face of the latching lug each forming an undercut. Preferably, two latching lugs engage in a corresponding manner behind the transverse leg of the approximately U-shaped latching hook. The latching lugs and/or the latching hook can have a run-on bevel acting in a radial direction. This can also provide a firm and secure connection of the proximal end region of the support structure to the adapter.

Advantageously, it can be provided that the at least one latching lug is arranged on a proximal end region of the support structure, in particular on an outer face of the proximal end segment, and the at least one latching hook is arranged on a distal side of the adapter, it being possible for the latching hook to be attached via the longitudinal leg on the distal side of an annular section of the adapter. The proximal side of the approximately L-shaped or U-shaped transverse leg forms the undercut of the transverse leg against which the distal end face of the latching lug or latching lugs bears after the latching connection has been closed. The run-on bevel is preferably arranged on the latching lug and is designed sloping down in the proximal direction, i.e. with a decreasing radius in the proximal direction. As a result, assembly can be further simplified and, at the same time, it is possible to achieve further increased safety of the connection of the support structure to the adapter.

In a particularly advantageous manner, two approximately L-shaped latching hooks or one approximately U-shaped latching hook and two latching lugs interacting therewith can be arranged approximately symmetrically in the circumferential direction on both sides of the at least one pull element. If two pull elements acting against each other are provided, then in particular two pairs of L-shaped latching hooks and latching lugs, i.e. a total of four such latching hooks and four latching lugs, or two U-shaped latching hooks, each with two assigned latching lugs, are provided. This can ensure that the latching connection is centered in relation to the longitudinal axis and also in the circumferential direction when latching takes place, as a result of which assembly can be further simplified. Alternatively, for example, three or five latching hooks can be provided, each with corresponding latching lugs, by which means centering can also be effected.

According to a further aspect of the present disclosure, which is also claimed independently of the characterizing feature of claim 1, a shaft for a flexible endoscope or for a flexible endoscopic instrument comprises a first shaft section and a second shaft section arranged on the distal side of the first shaft section, wherein the second shaft section can be angled by means of at least one pull element extending within the second shaft section, and wherein the second shaft section has a preferably integral, in particular one-piece support structure. According to this aspect of the disclosure, the shaft further comprises a head arranged on the distal side of the second shaft section, which head is designed in particular as a camera head in the case of a shaft for a flexible endoscope and in particular as an end effector with a tool in the case of a shaft for a flexible endoscopic instrument. Furthermore, according to this aspect of the disclosure, the head has a transverse wall forming at least one insertion opening which is continuous in the longitudinal direction and through which the at least one pull element is guided, wherein the at least one pull element is held distally on the transverse wall, and wherein a part of the insertion opening remaining in the transverse direction next to the pull element is at least partially filled and thus closed by a distal projection of the support structure. The insertion opening can be an opening within the transverse wall, or an opening which is formed between an edge of the transverse wall, in particular a recess in the transverse wall, and an outer wall of the head, which can be, for example, a cylindrical sleeve. The insertion opening comprises an insertion region and a holding region, wherein the at least one pull element runs through the holding region of the insertion opening and is held on the distal side of the transverse wall. The at least one pull element can be, for example, a pull wire, onto the distal end region of which an end sleeve is crimped, with which the pull wire is suspended on the distal side of the transverse wall. According to this aspect of the disclosure, the remaining part of the insertion opening, namely the insertion region, is at least partially filled and thus closed by the distal projection formed in the distal direction on the distal end region of the support structure, for example on a distal end segment of the support structure, for fixing the pull element.

This makes it possible in a simple and safe manner to manufacture the head separately from the support structure and to insert the at least one pull element, for example two pull wires acting against each other, into the already pre-assembled head, these being automatically secured by the projection against being pulled out upon attachment of the head to the support structure. This is particularly advantageous if the head is a camera head since, on account of the electronic components contained therein, such as the electronic image recorder and possibly LEDs for illuminating the cavity, it is preferably manufactured separately from the support structure and the first and second shaft sections and therefore has to be installed later.

Preferably, a distal end region of the support structure forms an approximately closed, in particular annularly closed, lateral surface with a proximal end region of the head, the cover tube of the second shaft section being connected to the head by gluing in the region of the distal edge of the lateral surface. For example, the cover tube can have its distal end face glued in abutment onto a proximal side of the head, to which the lateral surface attaches, or onto a proximal end face of the sleeve that forms the outer wall of the head and that can end flush with the distal edge of the lateral surface, and/or can have its distal end region glued onto a distal end region of the lateral surface. In particular, the lateral surface has an axial extent of approximately 1.5 mm or more, which makes it possible for an adhesive point, where an adhesive is introduced between the cover tube and the head or the lateral surface, to be sufficiently far away from a proximal edge of the lateral surface such that penetration of adhesive into the interior of the head or the support structure can be safely avoided. In particular, the proximal end region of the head can be inserted into the distal end segment of the support structure in such a way that a substantially annularly closed lateral surface with a sufficient axial extent is formed. In this way, process reliability can be further increased, and a largely smooth transition of the outer surface of the shaft between the head and the second shaft section can be achieved by appropriate selection of the external diameter of the cover tube and of the cover of the head.

A further aspect of the present disclosure relates to a method for assembling a shaft for a flexible endoscope or for a flexible endoscopic instrument. For assembling a shaft for a flexible endoscope, a support structure, an adapter and a head designed as a camera head are made available, each of which is designed as described above; in the case of a flexible endoscopic instrument, a suitably designed end effector is made available as the head. Furthermore, at least one pair of pull wires is provided.

End sleeves are crimped onto the distal ends of the pull wires, and the distal ends of the pull wires are inserted with the end sleeves in the axial direction through the corresponding insertion openings of the head and are hooked behind the transverse wall. The distal end segment of the support structure is then attached to the head from the proximal direction, in such a way that the distal projections of the support structure engage in the insertion openings of the head and at least partially fill them, so that the pull wires with the end sleeves latch into place behind the transverse wall and are thereby fixed in the head. The adapter is attached to the proximal end segment from the proximal direction, with the latching connection being closed by latching engagement of the latching hooks and latching lugs.

The pull wires have preferably already been routed previously on or through the support structure and through the adapter. Likewise, further elements, such as electrical lines or a casing of a working channel, can be routed through the support structure and the adapter or have already been routed through them. Furthermore, it can be provided that a cover tube of the second shaft section is pulled over this. The cover tube can be glued in the region of the distal edge of the annular surface formed by the transverse wall of the camera head and the distal end segment, for example its distal end face can be glued in abutment onto a proximal side of the camera head, with the adhesive point maintaining a sufficient distance from the proximal edge of the distal end segment. Furthermore, Bowden cable sheaths, in which the pull wires are guided in the first shaft section, can be inserted into the adapter and fixed there, which step can also have already taken place beforehand. A shaft tube of the first shaft section can be pushed onto the holding section of the adapter, and the cover tube can in particular be glued in abutment onto the distal end face of the shaft tube, whereby the corresponding adhesive point maintains a sufficient distance from the distal edge of the outer surface of the adapter, in particular a base of the adapter.

To assemble a flexible endoscope or a flexible endoscopic instrument, a handle of the endoscope or a grip of the endoscopic instrument can be attached to the proximal end of the first shaft section, i.e. to the proximal end of the shaft tube, and can be connected to the pull wires for bending the controllable second shaft section.

The present disclosure further relates to a flexible endoscope and a flexible endoscopic instrument. The flexible endoscope or endoscopic instrument according to the disclosure has a shaft which is designed as described above. The flexible endoscope can have a camera head designed as described above, or the flexible endoscopic instrument can have a correspondingly designed end effector, which is arranged at the distal end of the second shaft section. Furthermore, the flexible endoscope can comprise a handle, or the flexible endoscopic instrument can comprise a grip, which handle or grip is permanently or releasably connected to a proximal end of the shaft. To control the second shaft section, the handle or the grip has, for example, one or more handwheels or rotary knobs, by means of which the at least one pull element can be actuated. Furthermore, the handle or the grip can carry additional operating elements. The flexible endoscope is in particular a medical endoscope, and the endoscopic instrument is in particular a surgical endoscopic instrument. The flexible endoscope or the flexible endoscopic instrument is preferably intended for one-time use, i.e. designed as a disposable endoscope or as a disposable instrument.

It will be appreciated that the features mentioned above and the features yet to be explained below are applicable not only in the respectively specified combination but also in other combinations or on their own, without departing from the scope of the present disclosure.

Further aspects of the disclosure will become clear from the following description of preferred exemplary embodiments and from the attached drawing.

### BRIEF DESCRIPTION OF DRAWINGS

FIG. 1 shows a flexible endoscope having a shaft according to a first exemplary embodiment of the disclosure;

FIG. 2 shows a distal end section of the shaft of the endoscope according to FIG. 1 in an oblique view, with a shaft tube of the first shaft section and a cover tube of the second shaft section having been removed;

FIGS. 3 and 4 show the distal end region of the first shaft section and the proximal end region of the second shaft section of the shaft according to FIG. 2, each in an oblique view;

FIGS. 5 and 6 show the camera head of the endoscope according to FIG. 1;

FIGS. 7 and 8 show the distal end region of the first shaft section and the proximal end region of the second shaft section of the shaft according to a second exemplary embodiment of the disclosure, each in an oblique view;

FIGS. 9 and 10 show the distal end region of the first shaft section and the proximal end region of the second shaft section of the shaft according to a third exemplary embodiment, each in an oblique view;

FIG. 11 shows a variant of the first to third exemplary embodiments.

### DETAILED DESCRIPTION OF PREFERRED EMBODIMENTS

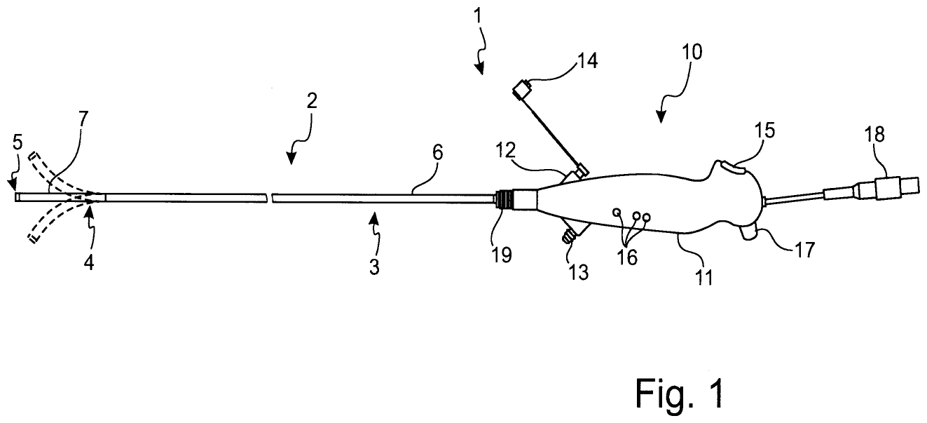

FIG. 1 shows an overall view of a first exemplary embodiment of a flexible endoscope 1 according to the disclosure. The endoscope 1 has an elongate flexible shaft 2, which is designed in particular for insertion into an internal cavity of a human or animal body. The shaft 2 comprises a first shaft section 3, at the distal end of which a second shaft section 4 is arranged. The first shaft section 3 is flexible and can therefore, for example, adapt to the shape of a curved access path to a cavity inside the body. As is indicated in FIG. 1, the second shaft section 4 is controllable and can be curved, at least in one plane, from an elongate position to a position angled to one side or the other. A camera head 5 is arranged at the distal end of the second shaft section 4. An outer surface of the first shaft section 3 is formed by a flexible shaft tube 6 and an outer surface of the second shaft section 4 is formed by a cover tube 7, which transition substantially smoothly into each other.

At the proximal side, the shaft 2 is connected to a handle 10, which serves to operate the endoscope 1 and for this purpose has an ergonomically shaped housing 11 on which operating elements and connections for further devices are arranged. In particular, the housing has an access port 12 for inserting a working instrument into a working channel that extends to the camera head 5, and also a hose connector 13 for connecting a suction hose, the access port 12 being closable with a closure cap 14 held on a flexible tab. Furthermore, a push button 15 for controlling the suction and a plurality of operating elements 16 for controlling other functions of the endoscope, such as light and zoom, are arranged on the housing 11.

Furthermore, the handle 10 comprises a control mechanism for bending the controllable second shaft section 4, which control mechanism can be actuated by means of a lever 17. Furthermore, an electrical connection 18 is connected to the housing 11 via a cable, which is longer than depicted and which serves for connecting the endoscope 1 to a supply and evaluation device (not shown), in order to supply electrical and electronic components of the endoscope 1 with electrical energy and to display and process a recorded endoscopic image. In the distal end region of the handle 10, in which the first shaft section 3 is connected to the housing 11, a plurality of retaining lips 19 are arranged, which securely hold the endoscope 1 during use in an insertion instrument.

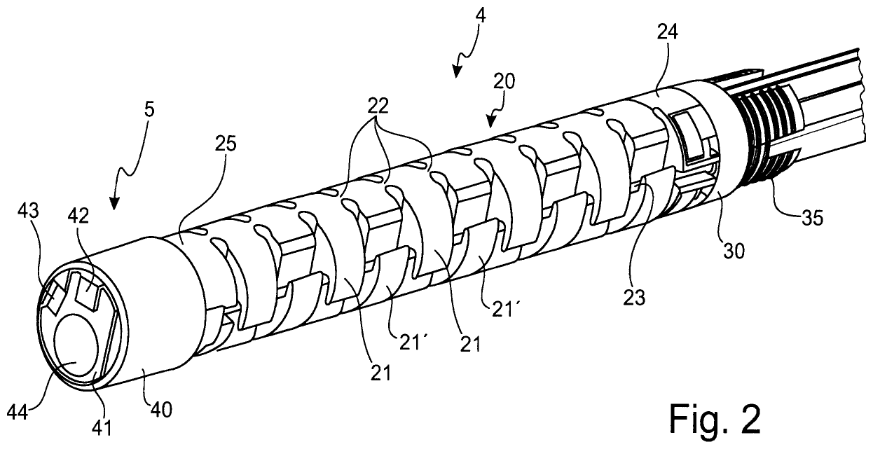

In FIG. 2, the second shaft section 4 is shown in an enlarged view, seen obliquely from the distal direction. The cover tube 7 and the proximally adjoining shaft tube 6 of the first shaft section 3 (see FIG. 1) are not shown in FIG. 2. As is shown in FIG. 2, the second shaft section 4 has a support structure 20, which is formed in one piece. The support structure 20 is formed by a plurality of links or segments which are arranged adjoining one another and which are connected to one another by webs 22, namely a plurality of middle segments 21, 21′ and also a proximal end segment 24 and a distal end segment 25. The webs 22 are each flexible and form film hinges through which two successive segments 21, 21′, 24, 25 can be pivoted against each other, so that the support structure 20 and thus the second shaft section 4 overall can assume a curved and therefore angled shape. To control the bending, two pull wires are routed through the second shaft section 4 and arranged approximately symmetrically to a longitudinal axis of the second shaft section 4 and to a central plane defined by the pivot axes of the film hinges formed by the webs 22. Of the pull wires arranged symmetrically on both sides of the central plane, only the front pull wire 23 can be seen in FIG. 2. The immediately adjacent middle segments 21, 21′ are designed differently from each other in order to improve the assembly and guiding of the pull wires 23. In particular, the middle segments 21, 21′ of the support structure 20 can be designed as in the German patent application DE 10 2020 134 603.4 (not a prior application) (internal file number: P20043DE), which in this regard is incorporated by reference into the present application. As will be explained in detail below, the proximal end segment 24 is designed to be connected to an adapter 30 which establishes the connection to the proximal shaft section 3, and the distal end segment 25 is designed to be connected to the camera head 5.

In FIG. 2, the support structure 20 is shown in the elongate arrangement, in which the second shaft section 4 is arranged in a straight continuation of the distal end region of the first shaft section 3 and in which a longitudinal axis of the second shaft section 4 is in alignment with a longitudinal axis of the distal end region of the first shaft section 3. In this arrangement, the central axes of the middle segments 21, 21′ and of the end segments 24, 25 and of the adapter 30 are also aligned with one another.

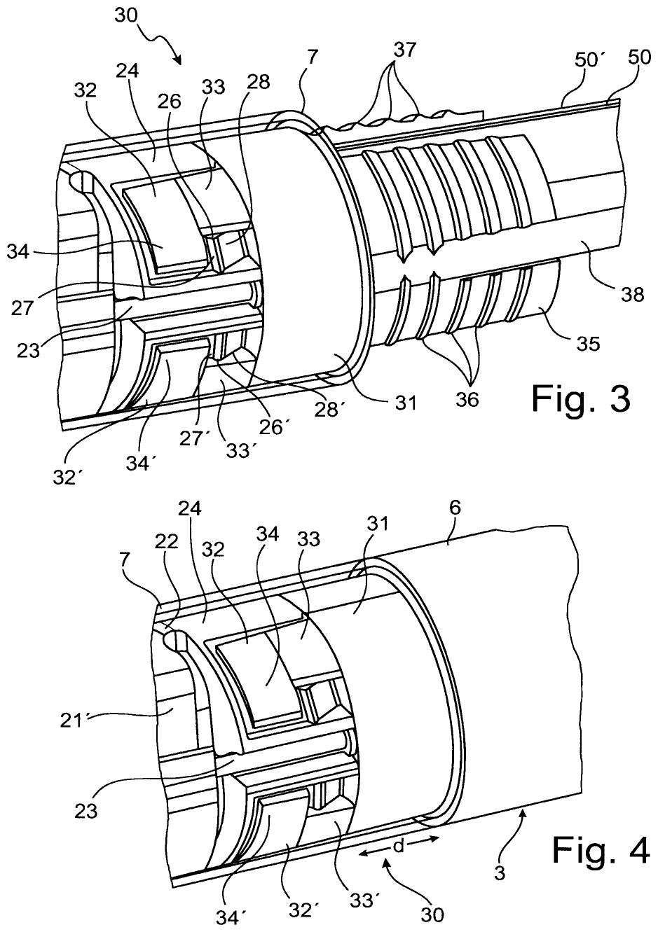

In FIGS. 3 and 4, the proximal end region of the second shaft section 4 and the distal end region of the first shaft section 3 are shown enlarged. As is shown in FIG. 4, the outer surface of the first shaft section 3 is formed by the shaft tube 6; the shaft tube 6 is not shown in FIG. 3. The outer surface of the second shaft section 4 is formed by the cover tube 7, which is shown transparently in FIGS. 3 and 4. The shaft tube 6 and the cover tube 7 connect to each other largely without any gaps; any gap that may be present is sealed tightly by gluing, so that a smooth outer surface of the shaft 2 is formed.

As can be seen in FIG. 3, the proximal end segment 24 has latching lugs 26, 26′ on its outside, each of which latching lugs has a distal end face 27, 27′ and a run-on bevel 28, 28′. The adapter 30 comprises an annular base 31, on the distal side of which L-shaped latching hooks 32, 32′ are arranged. The latching hooks 32, 32′ each have a longitudinal leg 33, 33′, which is directed in the longitudinal direction and attaches to the base 31, and a transverse leg 34, 34′, which extends in the circumferential direction and, on its proximal side, forms an undercut behind which the corresponding latching lug 26, 26′ engages. In total, four latching hooks 32, 32′ are provided on the base 31, and four corresponding latching lugs 26, 26′ are provided on the proximal end segment 24, which are arranged in pairs symmetrically to the pull wires 23.

During assembly of the shaft, the support structure 20 is connected to the adapter 30 by virtue of the proximal end segment 24 of the support structure 20 and the adapter 30 adjoining each other in the longitudinal direction. The latching hooks 32, 32′ slide with their transverse legs 34, 34′ over the run-on bevels 28, 28′ and latch behind the end faces 27, 27′ of the latching lugs 26, 26′. The latching hooks 32, 32′ are pressed radially outward in order to overcome the latching lugs 26, 26′. The latching hooks 32, 32′ are therefore deformed radially outward from the central axis of the proximal end segment 24; a relatively slight deformation may suffice to overcome the latching lugs 26, 26′. The surface of the undercut of the transverse leg 34, 34′ that interacts with the respective end face 27, 27′ for holding the proximal end segment 24 in the axial direction on the adapter 30 extends in the circumferential direction, such that a relatively large surface area is available for this despite the deformation being limited in the radial direction on account of the small size ratios. It is thereby possible to achieve a secure connection of the support structure 20 to the adapter 30. By virtue of the fact that the latching hooks 32, 32′ and the latching lugs 26, 26′ are arranged symmetrically to the pull wires 23, the adapter 30 and the proximal end segment 24 can be automatically centered relative to each other when they latch into place.

As is also shown in FIG. 3, the adapter 30 further comprises a holding section 35 which tapers conically in the proximal direction and which is attached to the proximal side of the annular base 31, and on the surface of which a plurality of holding ribs 36 are arranged, each of them having an outwardly directed cutting edge 37. The holding ribs 36 can have an asymmetrical profile, such that the cutting edges 37 are directed obliquely in the distal direction. The shaft tube 6 of the first shaft section 3 can be pushed onto the insertion section 35 of the adapter (see FIG. 4), with the holding section 35 being prevented from being pulled out of the shaft tube 6 by the holding ribs 36, which cut into an inner face of the shaft tube 6.

While the support structure 20 has to be sufficiently flexible and therefore consists of a material with a relatively low modulus of elasticity, it is advantageous for the adapter 30 to be made of a stiffer material, so that the holding section 35 can build up a sufficient counter-tension to the shaft tube 6 in order to hold the latter firmly. It is advantageous for the shaft tube 6 to be stiffer than the second shaft section 4, in order to be able to insert the first shaft section 3 through a curved access path and to be able to controllably angle the second shaft section 4 relative to the distal end region of the first shaft section 3. The adapter 30 is therefore made of a stiffer material than the support structure 20. For example, the support structure 20 can consist of a polypropylene with an elastic modulus of approximately 1400 MPa, and the adapter can consist of a polycarbonate with an elastic modulus of approximately 2400 MPa. In particular, the adapter 30 with the latching hooks 32, 32′ and the holding section 35 is made in one piece from the same material.

As is also indicated in FIG. 3, the pull wire 23 is guided in a Bowden cable sheath 38 on the proximal side of the adapter 30; the pull wire 23 interacts with the Bowden cable sheath 38 in the manner of a Bowden cable, in order to control the bending of the second shaft section 4 by means of a longitudinal movement of the pull wire 23 relative to the Bowden cable sheath 38. The Bowden cable sheath 38 is fixed in the annular base 31 and can bear with a distal end face on a proximal end face of the proximal end segment 24. Furthermore, the Bowden cable sheath 38 runs through a recess in the holding section 35 and can be secured there by adhesive.

The cover tube 7 is pulled over the support structure 20 and over the approximately cylindrical outer surface of the annular base 31 of the adapter 30. Furthermore, the proximal end face of the cover tube 7 is glued to the distal end face of the shaft tube 6, which is pushed onto the insertion section 35 of the adapter 30 as far as a proximal end face of the base 31 (see FIG. 4). The base 31 has a sufficient axial extent d such that the adhesive point, or a region in which adhesive can penetrate between the shaft tube 6 and the outer surface of the base 31, is sufficiently far away from the distal edge of the base 31 to prevent penetration of adhesive into the interior of the adapter 30 or between the pull wire 23 and the Bowden cable sheath 38. For this purpose, for example, the cylindrical outer surface of the base 31 can have an axial extent d of approximately 2 mm.

As is also shown in FIG. 2, the camera head 5 is approximately cylindrical, wherein a radially outer surface of the camera head is formed by a cover 40 which, like the cover tube 7 and the shaft tube 6, preferably consists of a plastic material. A window 42 for imaging optics and a further window 43 for illumination optics are indicated in a distal end surface 41 of the camera head 5. The working channel 44 also opens into the distal end surface 41 and runs continuously from the access port 12 on the handle 10 through the first and second shaft sections 3, 4 and opens out in the distal end surface 41. Through the working channel 44, working instruments, which are preferably flexible but not able to be actively angled, can be guided beyond the distal end of the endoscope 1 in order to carry out manipulations in the cavity.

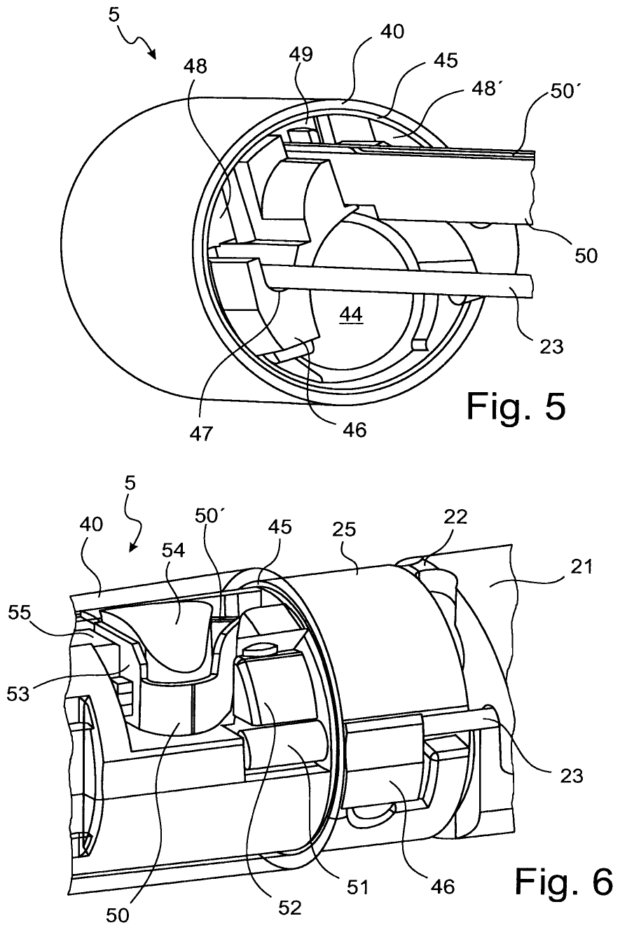

In FIG. 5, the camera head 5 is shown obliquely from the proximal direction. The camera head 5 has an approximately cylindrical sleeve 45, which at the same time forms the cover 40 or is a single component with the latter. At the proximal end of the camera head 5, a transverse wall 46 is arranged which is designed like a ring segment and partially encloses the working channel 44. On the inside, the transverse wall can have axially extending reinforcing ribs (not shown). An insertion opening 48 is formed between a recess 47 in the transverse wall 46 and the sleeve 45, through which insertion opening 48 the pull wire 23 is routed in the distal direction into the camera head 5. A corresponding further insertion opening 48′ for the second pull wire (not visible in FIG. 5) arranged on an opposite side of the central plane is arranged symmetrically to the insertion opening 48 with respect to the central plane. Approximately in the central plane, electrical lines are routed into the camera head 5 through a further opening 49, for example in the form of two ribbon cables 50, 50′ or flexible circuit boards. These run through the first and second shaft sections 3, 4 and form an electrical connection of the camera head 5 to the handle 10 (see FIGS. 1 and 3).

FIG. 6 shows a proximal region of the camera head 5 and the distal end segment of the support structure 20 in a view seen obliquely from the distal direction, in which the cover 40 and the sleeve 45 are shown transparently, and the cover tube 7 of the second shaft section 4 is not shown. To connect the camera head 5 to the support structure 20, the camera head 5 is inserted with the transverse wall 46 into the distal end segment 25. The distal end of the pull wire 23 is fixed in a crimped-on end sleeve 51, which is suspended on the distal side in the recess 47 of the transverse wall 46 (see FIG. 5). As is shown in FIG. 6, the end sleeve 51 can be approximately cylindrical, but also, for example, cuboid. The distal end segment 25 has a distal projection 52 which is fitted into the insertion opening 48 and fills the latter and thereby closes it, such that the end sleeve 51 with the pull wire 23 is prevented from slipping out. In a corresponding manner, the second pull wire arranged symmetrically opposite is fixed on the distal side of the transverse wall 46, the corresponding insertion opening 48′ being closed by an opposite projection arranged symmetrically to the projection 52. In this way, the camera head 5 is held firmly on the support structure 20.

FIG. 6 also shows that the ribbon cable 50 is connected on the distal side to an electronic image recorder, for example an image sensor 53 designed as a CCD or CMOS chip, which bears on a distal side of a cross-member 54. The ribbon cable 50 is guided around the cross-member 54, which in this way also acts as a tension relief. On a sensor surface of the image sensor 53, an image of a scene arranged distally with respect to the end surface 41 is produced by the imaging optics 55 indicated in FIG. 6. The image recorded by the image sensor 53 is forwarded via the ribbon cable 50 to an evaluation and display device that can be connected via the connection 18. The further ribbon cable 50′ serves to supply a light source, for example one or more light-emitting diodes (LED), with electrical energy.

The distal end of the cover tube 7 (not shown in FIG. 6) of the second shaft section 4 is fastened by gluing to the distal end segment 25 and/or to the camera head 5, the distal end face of the cover tube 7 being glued to a proximal end face of the cover 40 or the sleeve 45. The adhesive point is therefore arranged in the region of the distal edge of the lateral surface formed by the distal end segment 25 together with an outer face of the transverse wall 46, such that it is possible to reliably rule out any penetration of adhesive when gluing the cover tube 7 into the interior of the distal end segment 25. For this purpose, it may be sufficient, for example, for the transverse wall 46 to protrude in the axial direction with a thickness of approximately 1.5 mm on the proximal side beyond the sleeve 45 and the cover 40.

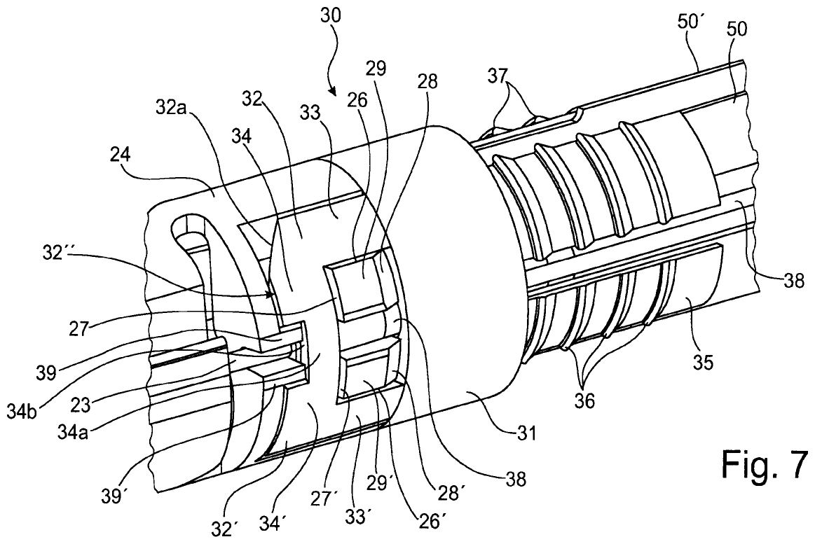

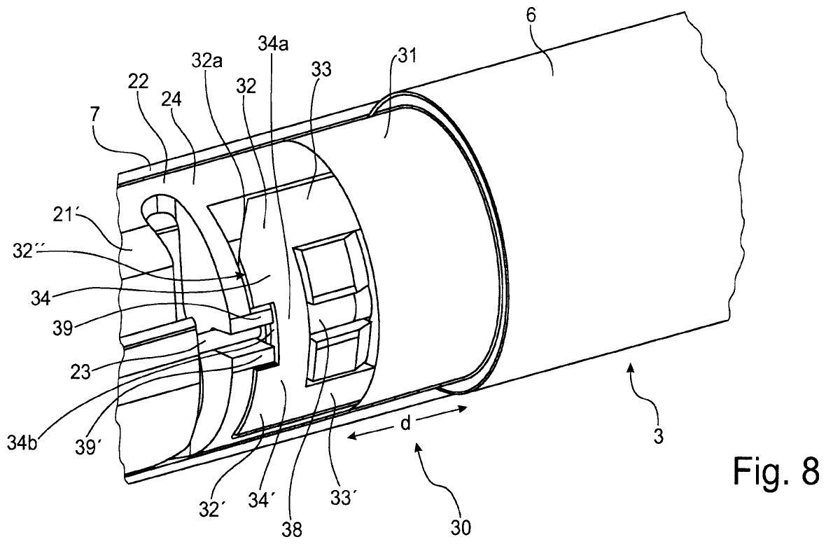

FIGS. 7 and 8 show a second exemplary embodiment of the disclosure; the shaft tube 6 of the first shaft section 3 and the cover tube 7 of the second shaft section 4 are not shown in FIG. 7, while the shaft tube 6 pulled over the holding section 35 is shown in FIG. 8 and the cover tube 7 is indicated transparently.

According to the second exemplary embodiment, the transverse legs 34, 34′ of the L-shaped latching hooks 32, 32′, which are arranged at least substantially symmetrically to the pull wire 23, are connected to each other via a bridge 34a extending in the circumferential direction and thereby form an approximately U-shaped latching hook 32″. In particular, each pair of latching hooks 32, 32′ arranged symmetrically to the pull wires 23 can form such a U-shaped latching hook 32″. The bridge 34a is narrower in the longitudinal direction of the adapter 30 than the transverse legs 34, 34′, and the proximal end segment 24 of the support structure 20 also carries lugs 39, 39′ which are arranged symmetrically to the pull wire 23 and which engage in the recess 34b formed on the distal side of the bridge 34a. This can result in improved centering, and securing against rotation, of the adapter 30 relative to the proximal end segment 24. As is indicated in FIGS. 7 and 8, the Bowden cable sheath 38 can extend below the bridge 34a. As is likewise indicated in FIGS. 7 and 8, at least on one latching hook 32, the distal, circumferentially outer corner can have a bevel 32a in order to facilitate the connection of the adapter 30 to the support structure 20.

In the second exemplary embodiment, shown in FIGS. 7 and 8, the latching lugs 26, 26′ are also longer in the longitudinal direction of the adapter 30 than in the first exemplary embodiment, in which the run-on bevels 28, 28′ adjoin the end faces 27, 27′ (see FIGS. 3 and 4), and they each have a flat plateau 29, 29′ on the top between the end face 27, 27′ and the run-on bevel 28, 28′. As a result, an increased load capacity of the connection of the adapter 30 to the support structure 20 can be achieved. Furthermore, the holding section 35 of the adapter 30 is provided with holding ribs 36, as in the second exemplary embodiment, but is not designed to taper conically.

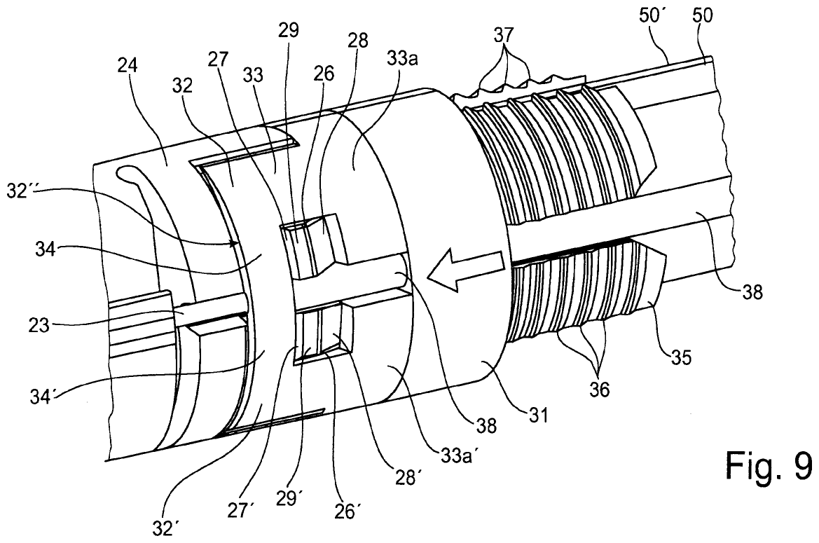

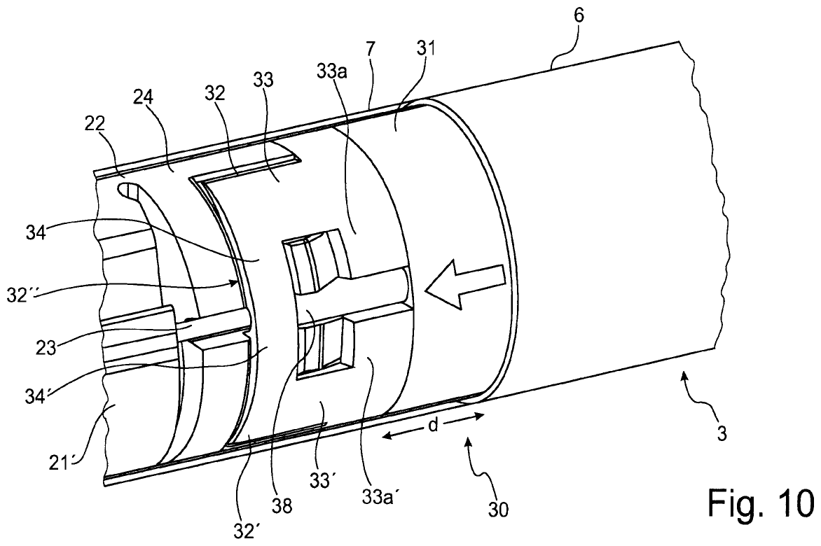

A third exemplary embodiment of the disclosure is shown in FIGS. 9 and 10, wherein, as in the second exemplary embodiment, the shaft tube 6 and the cover tube 7 are not shown in FIG. 9, but are shown or transparently indicated in FIG. 8.

According to the third exemplary embodiment, the transverse legs 34, 34′ of the L-shaped latching hooks 32, 32′, which are arranged symmetrically to the pull wire 23, are extended in the circumferential direction and connected directly to each other. The latching hooks 32, 32′ thus also form an approximately U-shaped latching hook 32″. In particular, each pair of latching hooks 32, 32′ arranged symmetrically to the pull wires 23 can be designed as such an approximately U-shaped latching hook 32″. The Bowden cable sheath 38 can extend below the interconnected transverse legs 34, 34′. The longitudinal legs 33, 33 ‘of the U-shaped locking hook 32″ each have a base section 33a, 33a’, which is designed as a ring segment extending in the circumferential direction and is connected to the base 31 of the adapter 30, with the Bowden cable sheath 38 running in a gap between the two base sections 33a, 33a′. As is indicated in FIGS. 9 and 10, the latching lugs 26, 26′ can likewise be designed longer in the longitudinal direction of the adapter 30 than in the first exemplary embodiment (see FIGS. 3 and 4) and have a plateau 29, 29′ on the top. In this way, too, an increased load capacity of the connection of the adapter 30 to the support structure 20 can be achieved. In FIGS. 9 and 10, the arrow applied to the base 31 of the adapter 30 indicates the direction in which the adapter 30 is moved relative to the proximal end segment 24 of the support structure 20 in order to establish the latching connection.

Otherwise, the second and third exemplary embodiments are designed like the first exemplary embodiment shown in FIGS. 1 to 6 and described above. In particular, the endoscope 1 according to the second and third exemplary embodiments can be designed as shown in FIG. 1 and the camera head can be designed as shown in FIG. 2 or FIGS. 5 and 6. Furthermore, in particular in the second and third exemplary embodiments, the shaft 2 or the endoscope 1 can be assembled as described above.

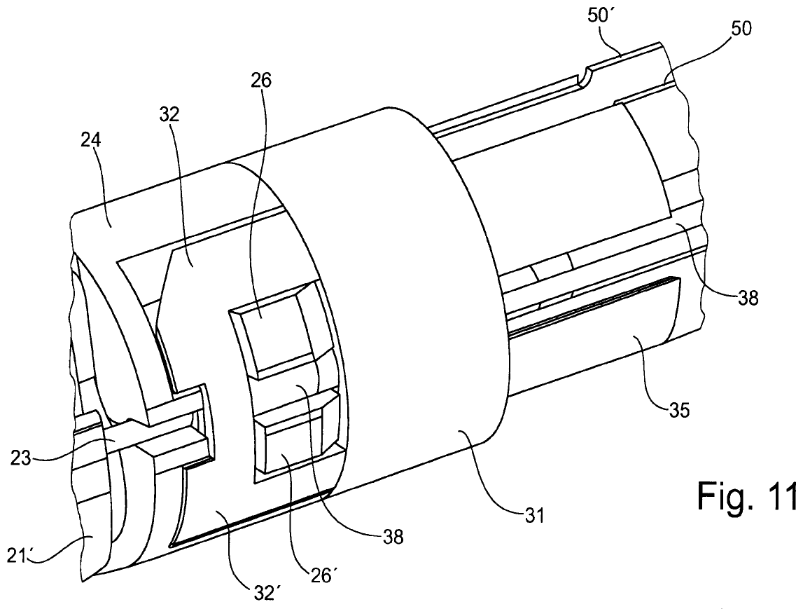

FIG. 11 shows a variant which differs from the second exemplary embodiment only in that the holding section 35 is smooth on the outside and in particular has no holding ribs 36 (see FIG. 7). As is shown in FIG. 8, the shaft tube 6 can be pulled over the holding section 35; here, the shaft tube 6 can be connected to the adapter 30 solely by friction or also by cohesive bonding such as gluing. In corresponding variants of the first and third exemplary embodiments, the holding section 35 can also be designed to be smooth on the outside. The variants mentioned can also be designed and assembled as described above.

According to a further exemplary embodiment of the disclosure, an endoscopic instrument comprises a shaft which can be designed like the shaft 2 of the flexible endoscope 1 according to the first to third exemplary embodiments, but, instead of the camera head 5, an end effector, which can have a tool for carrying out manipulations within the cavity, is arranged at the distal end of the second shaft section 4. The endoscopic instrument can further comprise a grip which is arranged at the proximal end of the shaft and which is designed, for example, like the handle 10 of the flexible endoscope 1 described above, wherein the grip can have an actuating element for actuating the tool.

For the sake of clarity, not all reference signs are shown in all of the figures. Reference signs not explained in the context of one figure have the same meaning as in the other figures.

1. A shaft for a flexible endoscope or for a flexible endoscopic instrument, the shaft comprising:
a first shaft section;
a second shaft section arranged on the distal side of the first shaft section, the second shaft section including at least one pull element, the at least one pull element extending inside the second shaft section and operable to angle the second shaft section with respect to the first shaft section, and wherein the second shaft section has a support structure; and
an adapter arranged between a distal end region of the first shaft section and a proximal end region of the support structure.

2. The shaft as claimed in claim 1, wherein the first shaft section can be passively deflected, and the support structure of the second shaft section is formed integrally, in particular in one piece, and includes a plurality of segments which follow one another in a longitudinal direction of the shaft and are articulated to one another.

3. The shaft as claimed in claim 1, wherein the pull element is a pull element of a Bowden cable, and a distal end of a sheath of the Bowden cable is fixed in the adapter.

4. The shaft as claimed in claim 1, wherein the adapter is at least partially annular with an approximately cylindrical outer surface, wherein a cover tube of the second shaft section in the region of a proximal edge of the approximately cylindrical outer surface is connected by gluing to the adapter and/or the first shaft section.

5. The shaft as claimed in claim 1, wherein the adapter has a proximal section which is inserted into a distal end region of a shaft tube of the first shaft section and on the surface of which one or more holding ribs are formed for holding the shaft tube or of which the surface is smooth.

6. The shaft as claimed in claim 1, wherein the support structure is made of a first material and the adapter is made of a second material, the second material having a greater modulus of elasticity than the first material.

7. The shaft as claimed in claim 1, wherein the adapter is connected to a proximal end region of the support structure by means of a latching mechanism which includes at least one latching hook and at least one latching lug cooperating therewith.

8. The shaft as claimed in claim 7, wherein the latching hook is approximately L-shaped with a longitudinal leg and with a transverse leg directed in the circumferential direction, the latching lug bearing on the transverse leg, and the latching lug and/or the latching hook having a run-on bevel acting in a radial direction.

9. The shaft as claimed in claim 7, wherein the latching hook is approximately U-shaped with two longitudinal legs and with a transverse leg directed in the circumferential direction, the at least one latching lug bearing on the transverse leg, and the latching lug and/or the latching hook having a run-on bevel acting in a radial direction.

10. The shaft as claimed in claim 8, wherein the latching lug is formed on a proximal end region of the support structure and the latching hook is formed on a distal side of the adapter, wherein a proximal side of the transverse leg forms an undercut, against which a distal end face of the latching lug bears.

11. The shaft as claimed in claim 7, wherein two approximately L-shaped latching hooks or one approximately U-shaped latching hook and two latching lugs interacting therewith are arranged approximately symmetrically on both sides of the at least one pull element.

12. The shaft as claimed in claim 1, wherein the shaft further includes a head arranged distally with respect to the second shaft section, wherein the head has a transverse wall forming at least one insertion opening which is continuous in the longitudinal direction and through which the at least one pull element is guided and held distally on the transverse wall, and wherein a part of the insertion opening remaining next to the pull element is at least partially filled by a distal projection of the support structure.

13. The shaft as claimed in claim 12, wherein a distal end region of the support structure forms an approximately closed lateral surface with a proximal end region of the head, a cover tube of the second shaft section being connected to the head by gluing in the region of a distal edge of the lateral surface.

14. A method for assembling a shaft for a flexible endoscope or for a flexible endoscopic instrument, which shaft includes a first shaft section and a second shaft section arranged on the distal side of the first shaft section, wherein the second shaft section can be angled by means of at least one pair of pull wires extending within the second shaft section, and wherein the second shaft section has a preferably integral, in particular one-piece support structure, with the steps:
making available the support structure of the second shaft section, an adapter, a head, and at least one pair of pull wires;
crimping end sleeves onto the distal ends of the pull wires and inserting the distal ends of the pull wires with the end sleeves through insertion openings of the head;
attaching a distal end segment of the support structure to the head, so that distal projections of the distal end segment engage in the insertion openings of the head and at least partially fill them, so that the pull wires are fixed in this; and
attaching the adapter from the proximal direction to a proximal end segment of the support structure, and locking the adapter to the support structure.

15. A flexible endoscope or flexible endoscopic instrument, wherein the flexible endoscope or the flexible endoscopic instrument includes a shaft according to claim 1.
